(19) United States
(12) Patent Application Publication
(10) Pub. No.: US 2026/0002128 A1
(43) Pub. Date: Jan. 1, 2026

(54) COMPOSITION AND METHOD FOR EX VIVO EXPANSION OF STEM CELL MEMORY T CELLS

(71) Applicant: LUKAS BIOMEDICAL INC., New Taipei City (TW)

(72) Inventors: Wei Hua CHU, Kaohsiung City (TW); Ting-Yun LIU, New Taipei City (TW); Pei-Yi LIN, Taichung City (TW)

(73) Assignee: LUKAS BIOMEDICAL INC., New Taipei City (TW)

(21) Appl. No.: 19/014,566

(22) Filed: Jan. 9, 2025

(30) Foreign Application Priority Data
Jun. 26, 2024 (TW) 113123817

(51) Int. Cl. C12N 5/0783; C12N 5/00

(52) U.S. Cl. CPC C12N 5/0636; C12N 5/0018; C12N 2500/33; C12N 2500/84; C12N 2501/2307; C12N 2501/2315; C12N 2523/00

(57) ABSTRACT

The composition and method for ex vivo expansion of stem cell memory T cells provided by the embodiments of the present disclosure can amplify and culture a cell population with a higher proportion of memory T stem cells, a higher combined proportion of memory T stem cells and central memory T cells, and a higher proportion of natural killer T cells compared to other processes. Additionally, the obtained cell population demonstrates excellent cytotoxic effects against bone cancer and pancreatic cancer cell lines, leading to the production of cell products with enhanced cell therapy efficacy.

### CROSS-REFERENCE TO RELATED APPLICATIONS

This non-provisional application claims priority under 35 U.S.C. § 119(a) on Patent Application No. 113123817 filed in Taiwan (R.O.C) on Jun. 26, 2024 the entire contents of which are hereby incorporated by reference.

### BACKGROUND

### 1. Technical Field

This disclosure relates to a composition and a method for expansion of T cells, particularly to a composition and a method for ex vivo expansion of stem cell memory T cells.

### 2. Related Art

Immunotherapy is a new trend in cancer treatment today. Cancer immunotherapy helps the immune system to kill cancer cells while enhancing immune responses to destroy tumors. In short, the mechanism of immunotherapy involves, isolating mononuclear cells from the blood, followed by ex vivo processes such as culturing, inducing, activating, and expanding these mononuclear cells to become functional immune cells, which are subsequently infused into the patient to fight cancer cells.

It is known that among immune cell populations, stem cell memory T cells (Tscm) have strong proliferative capacity and long-term persistence in the body. Compared to memory cells in other stages, stem cell memory T cells exhibit superior therapeutic effects against cancer. Therefore, improving the production efficiency of stem cell memory T cells is one of the key focus areas for both industry and academia.

### SUMMARY

The embodiments of the present disclosure provide a composition and a method for ex vivo expansion of CD3+CD45RO−CD197+CD95+ stem cell memory T cells, capable of expanding and culturing a cell population containing a high proportion of stem cell memory T cells, thereby improving the production efficiency of stem cell memory T cells.

One embodiment of the present disclosure provides a composition for ex vivo expansion of stem cell memory T cells, comprising:
25 U/mL to 50 U/mL of interleukin-7 (IL-7) or 0.192 ng/mL to 25 ng/mL of interleukin-7;
10 U/mL to 50 U/mL of interleukin-15 (IL-15) or 0.5 ng/mL to 25 ng/mL of interleukin-15;
1 mM to 10 mM of N-acetylcysteine (NAC); and
0% to 10% (v/v) of human platelet lysate (hPL).

Another embodiment of the present disclosure provides a method for ex vivo expansion of stem cell memory T cells, comprising:
culturing a first cell population using the aforementioned composition for ex vivo expansion of stem cell memory T cells to obtain a second cell population,
wherein the first cell population comprises CD3+ activated T cells, CD3+CD45RO−CD197+CD95+ stem cell memory T cells (Tscm), CD3+CD45RO+CD197+ central memory T cells (Tem), CD3+CD45RO+CD197− effector memory T cells (Tem), CD3+CD56+ natural killer T cells (NKT cells), and CD3+CD8+ cytotoxic T cells (Cytotoxic T cells),
the second cell population comprises CD3+ activated T cells, CD3+CD45RO−CD197+CD95+ stem cell memory T cells, CD3+CD45RO+CD197+ central memory T cells, CD3+CD45RO+CD197− effector memory T cells, CD3+CD56+ natural killer T cells, and CD3+CD8+ cytotoxic T cells, and the proportion of CD3+CD45RO−CD197+CD95+ stem cell memory T cells in the second cell population may be 35.6% or higher.

According to the composition and the method for ex vivo expansion of stem cell memory T cells in the embodiments of the present disclosure, the obtained cell population has a high proportion of stem cell memory T cells. Moreover, compared to the cell populations produced by using compositions disclosed in the prior art, the cell population produced by using the composition in the embodiments of present disclosure has a higher proportion of stem cell memory T cells, as well as an increased combined proportion of stem cell memory T cells and central memory T cells. In addition, the cell population produced by using the composition and the method for ex vivo expansion of stem cell memory T cells in the embodiments of the present disclosure also has a high proportion of activated T cells, natural killer T cells, and cytotoxic T cells. Further, compared to the cell populations produced by using compositions disclosed in the prior art, the cell population produced by using the composition in the embodiments of the present disclosure shows superior cytotoxic effects against cancer cells. Therefore, according to the composition and the method for ex vivo expansion of stem cell memory T cells in the embodiments of the present disclosure may culture a cell population with a high proportion of stem cell memory T cells, improve the production efficiency of stem cell memory T cells, and hold great potential for use in cancer treatment or be used as a raw material for chimeric antigen receptor T cell (CAR-T) therapy.

### BRIEF DESCRIPTION OF THE DRAWINGS

The present disclosure will become more fully understood from the detailed description given herein below and the accompanying drawings which are given by way of illustration only and thus not limitative of the present disclosure and wherein:

FIG. 1 shows an analysis of the memory T cell population proportions obtained after cultured with compositions containing different concentrations of N-acetylcysteine (NAC).

FIG. 2 shows an analysis of the memory T cell population proportions obtained after cultured with compositions containing different concentrations (% v/v) of human platelet lysate (hPL).

FIG. 3 shows an analysis of the memory T cell population proportions obtained after cultured with compositions with and without N-acetylcysteine (NAC) and human platelet lysate (hPL).

FIG. 4 shows a statistical analysis of the memory T cell population proportions obtained after cultured with the composition of an embodiments of the present disclosure.

FIG. 5 shows a flow cytometry analysis chart of the memory T cell population obtained after cultured with the composition of the embodiment of the present disclosure.

FIG. 6 shows the statistical analysis of the T cell population proportions obtained after cultured with the composition of the embodiment of the present disclosure.

FIG. 7 shows the flow cytometry analysis chart of the T cell population obtained after cultured with the composition of the embodiment of the present disclosure.

FIG. 8 shows the analysis of the memory T cell population proportions obtained after cultured with the composition of the embodiment of the present disclosure and the composition of the comparative embodiment.

FIG. 9 shows the flow cytometry analysis chart of the memory T cell population proportion obtained after cultured with the composition of the embodiment of the present disclosure and the composition of the comparative embodiment.

FIG. 10 shows the analysis of the T cell population proportions obtained after cultured with the composition of the embodiment of the present disclosure and the composition of the comparative embodiment.

FIG. 11 shows the flow cytometry analysis chart of the T cell population proportion obtained after cultured with the composition of the embodiment of the present disclosure and the composition of the comparative embodiment.

FIG. 12 shows a cytotoxic efficiency of the cells cultured with the composition of the embodiment of the present disclosure and the composition of the comparative embodiment against osteosarcoma cancer cell lines, HOS, at different times and different E:T ratio.

FIG. 13 shows the cytotoxic efficiency of the cells cultured with the composition of the embodiment of the present disclosure and the composition of the comparative embodiment against pancreatic cancer cell lines, BxPC3, at different times and different E:T ratio.

### DETAILED DESCRIPTION

In the following detailed description, the features and advantages of the present disclosure provide sufficient information for one skilled in the art to understand the technical content of the present disclosure and to practiced it. Furthermore, based on the content disclosed in this specification, the claims, and the drawings, one skilled in the art can easily comprehend the objectives and advantages related to the present disclosure. The following embodiments further explain the concepts of the present disclosure in detail but are not intended to limit the scope of the present disclosure in any way.

One embodiment of the present disclosure provides a composition for ex vivo expansion of stem cell memory T cells, comprising 25 U/mL to 50 U/mL of interleukin-7, 10 U/mL to 50 U/mL of interleukin-15, 1 mM to 10 mM of N-acetylcysteine, and 0% to 10% (v/v) of human platelet lysate.

Based on common unit conversions between activity units and weight units for interleukin-7 available on the market, 1 gram (g) of protein can correspond to 2 activity units (U) to 130 activity units. For example, Peprotech corporation provides a conversion formula where 1 ng=2 U, meaning that the content of interleukin-7 in the composition for ex vivo expansion of stem cell memory T cells of the present disclosure would be 12.5 ng/mL to 25 ng/mL. Miltenyi corporation provides a conversion formula where 1 ng=130 U, meaning that the content of interleukin-7 in the composition for ex vivo expansion of stem cell memory T cells of the present disclosure would be 0.192 ng/mL to 0.385 ng/mL. Accordingly, in the composition for ex vivo expansion of stem cell memory T cells of the present disclosure, the content of interleukin-7 can range from 0.192 ng/mL to 25 ng/mL, but the present disclosure is not limited thereto. In other embodiments, the content of interleukin-7 may range from 0.385 ng/mL to 25 ng/mL. In other embodiments, the content of interleukin-7 may range from 12.5 ng/mL to 25 ng/mL. In other embodiments, the content of interleukin-7 may range from 0.192 ng/mL to 0.385 ng/mL. In other embodiments, the content of interleukin-7 may range from 5 ng/mL to 50 ng/mL. In other embodiments, the content of interleukin-7 may range from 0.077 ng/mL to 0.769 ng/mL.

Based on common unit conversions between activity units and weight units for interleukin-15 available on the market, 1 gram (g) of protein can correspond to 2 activity units (U) to 20 activity units. For example, Peprotech corporation provides a conversion formula where 1 ng=2 U, meaning that the content of interleukin-15 in the composition for ex vivo expansion of stem cell memory T cells of the present disclosure would be 5 ng/mL to 25 ng/mL. Miltenyi corporation provides a conversion formula where 1 ng=20 U, meaning that the content of interleukin-15 in the composition for ex vivo expansion of stem cell memory T cells of the present disclosure would be 0.5 ng/mL to 2.5 ng/mL. Accordingly, in the composition for ex vivo expansion of stem cell memory T cells of the present disclosure, the content of interleukin-15 can range from 0.5 ng/mL to 25 ng/mL, but the present disclosure is not limited thereto. In other embodiments, the content of interleukin-15 may range from 2.5 ng/mL to 25 ng/mL. In other embodiments, the content of interleukin-15 may range from 5 ng/mL to 25 ng/mL. In other embodiments, the content of interleukin-15 may range from 0.5 ng/mL to 2.5 ng/mL. In other embodiments, the content of interleukin-15 may range from 12.5 ng/mL to 1.25 ng/mL.

In other embodiments, the composition for ex vivo expansion of stem cell memory T cells of the present disclosure may comprise 0.192 ng/mL to 25 ng/mL of interleukin-7, 0.5 ng/mL to 25 ng/mL of interleukin-15, 1 mM to 10 mM of N-acetylcysteine, and 0% to 10% (v/v) of human platelet lysate.

In other embodiments, in the composition for ex vivo expansion of stem cell memory T cells of the present disclosure, the concentration of human platelet lysate may range from 0% to 5% (v/v).

Another embodiment of the present disclosure provides a method for ex vivo expansion of stem cell memory T cells, comprising culturing a first cell population by using the aforementioned composition for ex vivo expansion of stem cell memory T cells to obtain a second cell population. The first cell population comprises CD3+ activated T cells, CD3+CD45RO−CD197+CD95+ stem cell memory T cells, CD3+CD45RO+CD197+ central memory T cells, CD3+CD45RO+CD197− effector memory T cells, CD3+CD56+ natural killer T cells, and CD3+CD8+ cytotoxic T cells. The second cell population comprises CD3+ activated T cells, CD3+CD45RO−CD197+CD95+ stem cell memory T cells, CD3+CD45RO+CD197+ central memory T cells, CD3+CD45RO+CD197− effector memory T cells, CD3+CD56+ natural killer T cells, and CD3+CD8+ cytotoxic T cells, wherein the second cell population has a high proportion of CD3+CD45RO−CD197+CD95+ stem cell memory T cells.

In the following, CD3+ activated T cells will be referred to as “activated T cells”, CD3+CD45RO−CD197+CD95+ stem cell memory T cells will be referred to as “stem cell memory T cells”,CD3+CD45RO+CD197+ central memory T cells will be referred to as “central memory T cells”, CD3+CD45RO+CD197− effector memory T cells will be referred to as “effector memory T cells”, CD3+CD56+ natural killer T cells will be referred to as “natural killer T cells”, and CD3+CD8+ cytotoxic T cells will be referred to as “cytotoxic T cells”.

The first cell population may be obtained by manufacturing from mononuclear cells. Mononuclear cells may be derived from peripheral blood, cord blood, or bone marrow blood. For example, first, peripheral blood is collected, diluted with phosphate-buffered saline (PBS), and purified by using a cell separation medium (Ficoll-Paque) to obtain mononuclear cells. Next, the peripheral blood mononuclear cells are cultured by using a basic culture medium, which may include, but is not limited to, DMEM, X-VIVO, TexMACS medium, RPMI-1640, or AIM-V. CD3, CD3+CD28, Dynabeads, T-Cell TransAct, or MACSiBead CD3/CD28 may be added thereto so as to activate T cells, along with the addition of interleukin-2 (IL-2), interleukin-7 (IL-7), interleukin-15 (IL-15), or interleukin-21 (IL-21). The manufacturing time for mononuclear cells may range from 6 to 10 days. In this way, the first cell population can be obtained after cultured mononuclear cells, and the first cell population may include activated T cells, stem cell memory T cells, central memory T cells, effector memory T cells, natural killer T cells, and cytotoxic T cells.

The second cell population may be obtained by culturing the first cell population. The culture medium used may include a basic culture medium and the composition for ex vivo expansion of stem cell memory T cells of the embodiments of the present disclosure. The basic culture medium may include, but is not limited to, DMEM, RPMI-1640, AIM V, TexMACS medium, or X-VIVO15. The culture temperature may be 37° C., the culture time may range from 3 to 6 days, and the air environment may be atmosphere +5% CO₂.

The second cell population obtained in this way may include activated T cells, stem cell memory T cells, central memory T cells, effector memory T cells, natural killer T cells, and cytotoxic T cells. Among them, the proportion of stem cell memory T cells in the second cell population may be 35.6% or higher. Additionally, the proportion of natural killer T cells in the second cell population may be 17.1% or higher, and the proportion of cytotoxic T cells may be 64.2% or higher, but the present disclosure is not limited thereto. In other embodiments, the average proportion of stem cell memory T cells in the second cell population may be 46.1% or higher, the average proportion of natural killer T cells may be 33.2% or higher, and the average proportion of cytotoxic T cells may be 84.2% or higher. For example, the proportion of stem cell memory T cells in the second cell population may range from 35.6% to 64.7%, the proportion of natural killer T cells may range from 17.1% to 55.8%, and the proportion of cytotoxic T cells may range from 64.2% to 93.3%.

The second cell population obtained by culturing with the composition for ex vivo expansion of stem cell memory T cells of the embodiments of the present disclosures may contain a high proportion of stem cell memory T cells, while also being rich in natural killer T cells and cytotoxic T cells, and may exhibit excellent cytotoxic effects against cancer cells.

In the following experiment, various compositions are used to culture the first cell population to obtain the second cell population, and spectral flow cytometry is used to analyze the cell population proportions in the second cell population. Specifically, mononuclear cells are first manufactured by using the aforementioned method, and after 6 to 10 days of manufacturing, the first cell population is obtained. Next, the first cell population is manufactured using compositions from each embodiment or comparative embodiment, and after 3 to 6 days of culture at 37° C., the second cell population is obtained. The required number of cells is then taken from the second cell population, and the cells are washed with phosphate-buffered saline (PBS). Fluorescent antibodies are then added for cell staining. The cells are washed twice with PBS, and spectral flow cytometry is used to analyze the cell population proportions.

### Embodiment 1: Concentration of N-acetylcysteine

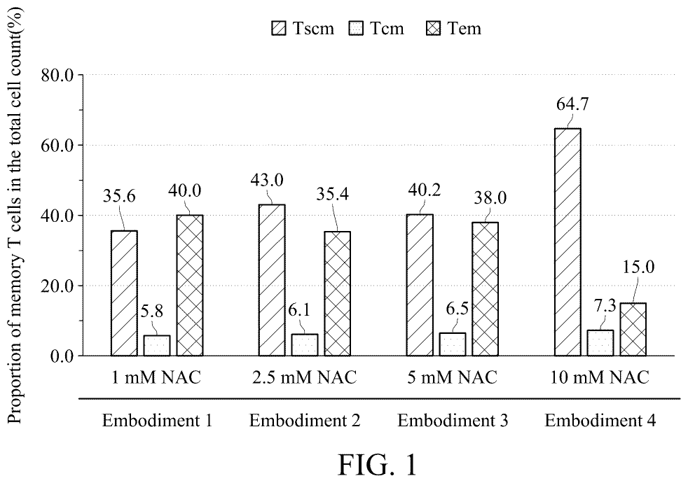

FIG. 1 shows the analysis of the memory T cell population proportions obtained after manufactured with compositions containing different concentrations of N-acetylcysteine (NAC). Table 1 discloses the components of the compositions used in each embodiment of this experiment. FIG. 1 and Table 2 show the proportions of stem cell memory T cells (Tscm), central memory T cells (Tcm), and effector memory T cells (Tem) in the activated T cell count of the second cell population. As shown in FIG. 1 and Table 2, after manufacturing the cells with the compositions of embodiments 1 to 4, which respectively contain 1 mM, 2.5 mM, 5 mM, and 10 mM of N-acetylcysteine, a high proportion of stem cell memory T cells (Tscm) is obtained, and the proportion of stem cell memory T cells (Tscm) increases as the concentration of N-acetylcysteine increased.

TABLE 1
Components of each composition

| Composition | Component | NAC | |
|---|---|---|---|
| Embodiment 1 | 50 U/mL interleukin-7 | 1 | mM |
| Embodiment 2 | 50 U/mL interleukin-15 | 2.5 | mM |
| Embodiment 3 | 5% (v/v) human platelet lysate | 5 | mM |
| Embodiment 4 | N-acetylcysteine (NAC) | 10 | mM |

TABLE 2
Proportion (%) of each type of memory
T cell in the activated T cell count

| | Tscm | Tcm | Tem |
|---|---|---|---|
| Embodiment 1 | 35.6 | 5.8 | 40.0 |
| Embodiment 2 | 43.0 | 6.1 | 35.4 |
| Embodiment 3 | 40.2 | 6.5 | 38 |
| Embodiment 4 | 64.7 | 7.3 | 15 |

### Embodiment 2: Concentration of Human Platelet Lysate

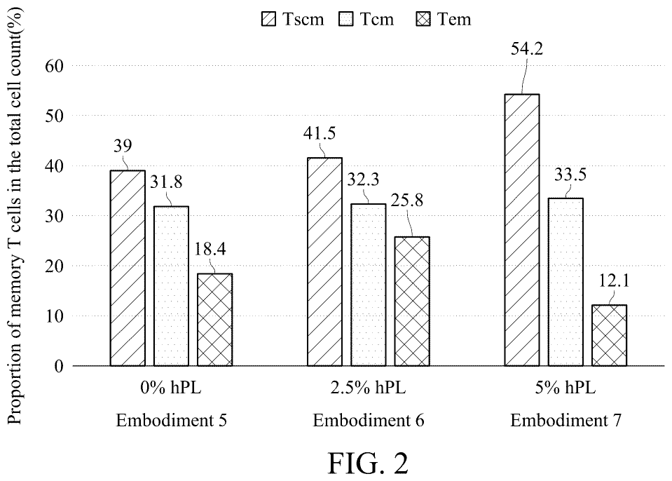

FIG. 2 shows the analysis of the memory T cell population proportions obtained after manufactured with compositions containing different concentrations (% v/v) of human platelet lysate (hPL). Table 3 discloses the components of the compositions used in each embodiment of this experiment. FIG. 2 and Table 4 show the proportions of stem cell memory T cells (Tscm), central memory T cells (Tcm), and effector memory T cells (Tem) in the activated T cell count of the second cell population. As shown in FIG. 2 and Table 4, after manufacturing the cells with the compositions of embodiments 5 to 7, which contain 0%, 2.5%, and 5% (v/v) of human platelet lysate, a high proportion of stem cell memory T cells (Tscm) is obtained, and the proportion of stem cell memory T cells (Tscm) increased as the concentration of human platelet lysate increased.

TABLE 3
Components of each composition

| Composition | Component | hPL | |
|---|---|---|---|
| Embodiment 5 | 50 U/mL interleukin-7 | 0% | v/v |
| Embodiment 6 | 50 U/mL interleukin-15 | 2.5% | v/v |
| Embodiment 7 | 10 mM N-acetylcysteine | 5% | v/v |

human platelet lysate (hPL)

TABLE 4
Proportion (%) of each type of memory
T cell in the activated T cell count

| | Tscm | Tcm | Tem |
|---|---|---|---|
| Embodiment 5 | 39 | 31.8 | 18.4 |
| Embodiment 6 | 41.5 | 32.3 | 25.8 |
| Embodiment 7 | 54.2 | 33.5 | 12.1 |

### Embodiment 3: Presence or Absence of N-Acetylcysteine and Human Platelet Lysate

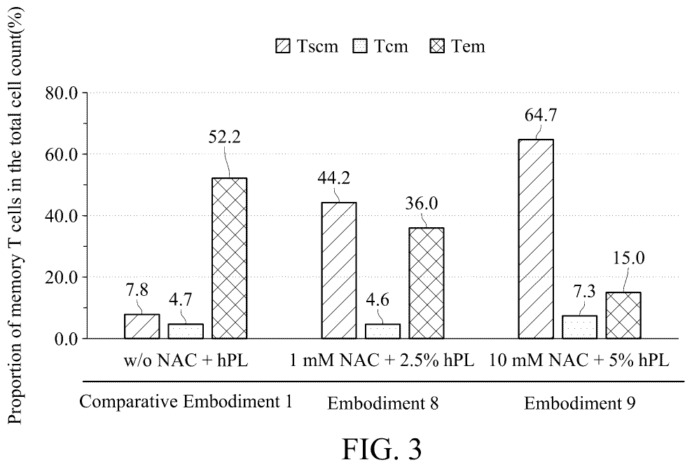

FIG. 3 shows the analysis of the memory T cell population proportions obtained after manufactured with compositions containing or not containing N-acetylcysteine (NAC) and human platelet lysate (hPL). Table 5 discloses the components of the compositions used in each embodiment or comparative embodiment of this experiment. FIG. 3 and Table 6 show the proportions of stem cell memory T cells (Tscm), central memory T cells (Tcm), and effector memory T cells (Tem) in activated T cell count of the second cell population. As shown in FIG. 3 and Table 6, compared to the composition of comparative embodiment 1, which does not contain N-acetylcysteine (NAC) or human platelet lysate (hPL), the composition of the embodiments containing N-acetylcysteine (NAC) and human platelet lysate (hPL) results in a higher proportion of stem cell memory T cells (Tscm).

TABLE 5
Components of each composition

| Composition | Component | NAC | | hPL | |
|---|---|---|---|---|---|
| Comparative | 50 U/mLinterleukin-7 | 0 | mM | 0% | v/v |
| Embodiment | 50 U/mLinterleukin-15 | | | | |
| 1 | N-acetylcysteine (NAC) | | | | |
| Embodiment | human platelet lysate (hPL) | 1 | mM | 2.5% | v/v |
| 8 | | | | | |
| Embodiment | | 10 | mM | 5% | v/v |

9

TABLE 6
Proportion (%) of each type of memory
T cell in the activated T cell count

| | Tscm | Tcm | Tem |
|---|---|---|---|
| Comparative Embodiment 1 | 7.8 | 4.7 | 52.2 |
| Embodiment 8 | 44.2 | 4.6 | 36.0 |
| Embodiment 9 | 64.7 | 7.3 | 15.0 |

### Experiment 4: Statistical Analysis of the Proportion of Memory T Cells

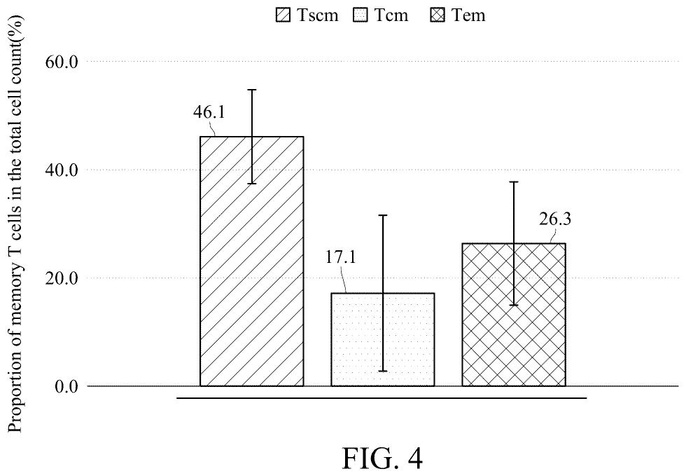

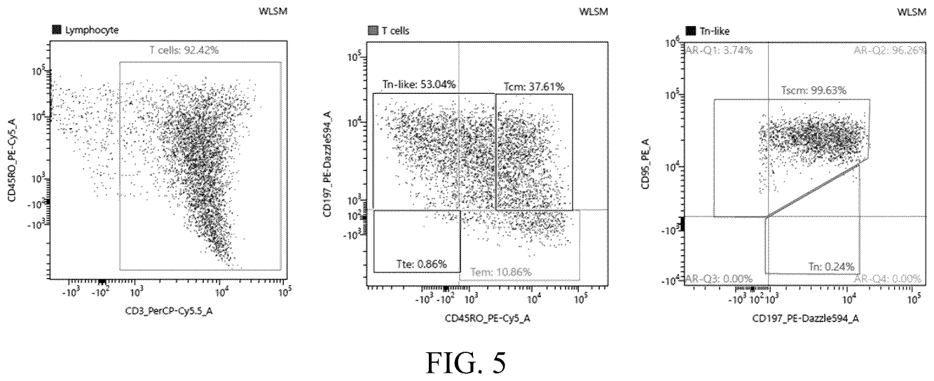

This experiment summarizes the proportion of memory T cells in the activated T cell count obtained from the aforementioned embodiments as well as other unshown embodiments, followed by statistical analysis. FIG. 4 shows the statistical analysis of the memory T cell population proportions obtained after manufactured with the composition of the embodiments of the present disclosure. FIG. 5 shows a flow cytometry analysis chart of the memory T cell population obtained after manufactured with the composition of the embodiment of the present disclosure. FIG. 4 and Table 7 show the statistical analysis of the proportions of stem cell memory T cells (Tscm), central memory T cells (Tcm), and effector memory T cells (Tem) in the activated T cell count of the second cell population. As shown in FIG. 4 and Table 7, after manufacturing cells with the composition of the embodiments of the present disclosure, a high proportion of stem cell memory T cells (Tscm) is obtained. Specifically, the proportion of stem cell memory T cells in the second cell population may be 35.6% or higher, and more specifically, it may range from 35.6% to 64.7%, with an average of 46.1% or higher.

TABLE 7
Statistical analysis of memory T cell population
proportions in the embodiments (%)

| | Tscm | Tcm | Tem |
|---|---|---|---|
| Average | 46.1 | 17.1 | 26.3 |
| Standard Deviation | 8.7 | 14.4 | 11.4 |
| Maximum Value | 64.7 | 37.6 | 40.0 |
| Minimum Value | 35.6 | 4.6 | 10.9 |

### Experiment 5: Statistical Analysis of the Proportions of Activated T Cells, Natural Killer T Cells, and Cytotoxic T Cells

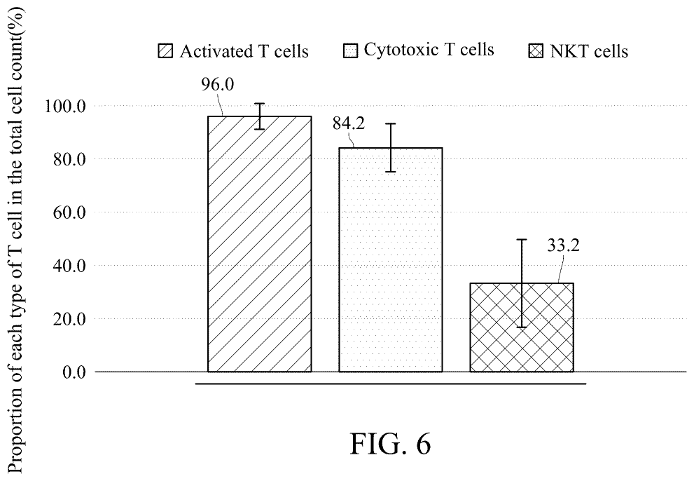

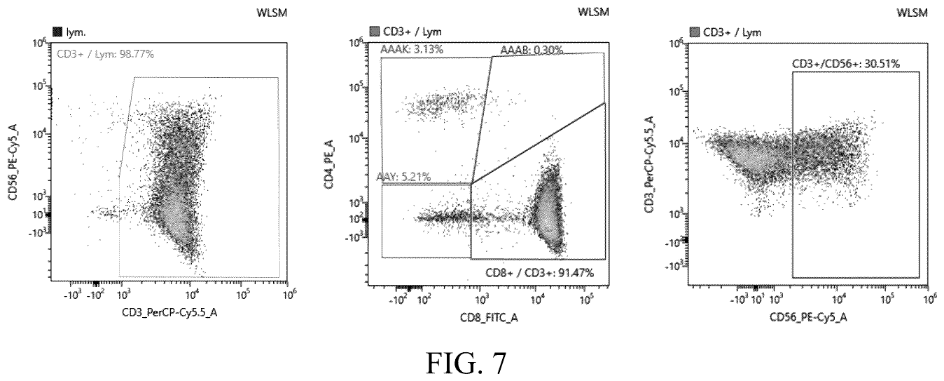

This experiment uses the composition of embodiment 10 for multiple batches of culture, and summarizes the proportions of various T cells in the total cell count from each unshown batch, followed by statistical analysis. FIG. 6 shows the statistical analysis of the cell population proportions obtained after cultured with the composition of the embodiment of the present disclosure. FIG. 7 shows the flow cytometry analysis chart of the cell population obtained after cultured with the composition of the embodiment of the present disclosure. Table 8 discloses the components of the composition of embodiment 10. FIG. 6 and Table 9 show the proportions and statistical analysis of CD3+ activated T cells (Activated T cells), CD3+CD56+ natural killer T cells (NKT cells), and CD3+CD8+ cytotoxic T cells (Cytotoxic T cells) in the second cell population across multiple batches. As shown in FIG. 6, FIG. 7, and Table 9, after culturing cells with the composition of the embodiment of the present disclosure, a high proportion of activated T cells, natural killer T cells, and cytotoxic T cells is obtained. Specifically, the proportion of activated T cells in the second cell population may be 87.9% or higher, and more specifically, it may range from 87.9% to 98.8%, with an average of 96.0% or higher. Specifically, the proportion of natural killer T cells in the second cell population may be 17.1% or higher, and more specifically, it may range from 17.1% to 55.8%, with an average of 33.2% or higher. Specifically, the proportion of cytotoxic T cells in the second cell population can be 64.2% or higher, and more specifically, it may range from 64.2% to 93.3%, with an average of 84.2% or higher.

TABLE 8
Component of the composition

| Composition | Component |
|---|---|
| Embodiment 10 | 50 U/mL interleukin-7 |
| | 50 U/mL interleukin-15 |
| | 10 mM N-acetylcysteine (NAC) |
| | 5% (v/v) human platelet lysate (hPL) |

TABLE 9
Proportion (%) of each type of T cell
population in the total cell count

| | Activated T | Cytotoxic | NKT |
|---|---|---|---|
| | Cells | T Cells | Cells |
| Average | 96.0 | 84.2 | 33.2 |
| Standard Deviation | 4.1 | 10.8 | 13.2 |
| Maximum Value | 98.8 | 93.3 | 55.8 |
| Minimum Value | 87.9 | 64.2 | 17.1 |

### Experiment 6: Comparison of the Proportion of Memory T Cells with Comparative Examples

The proportions of memory T cells in the activated T cell count from each unshown batch were summarized and statistically analyzed.

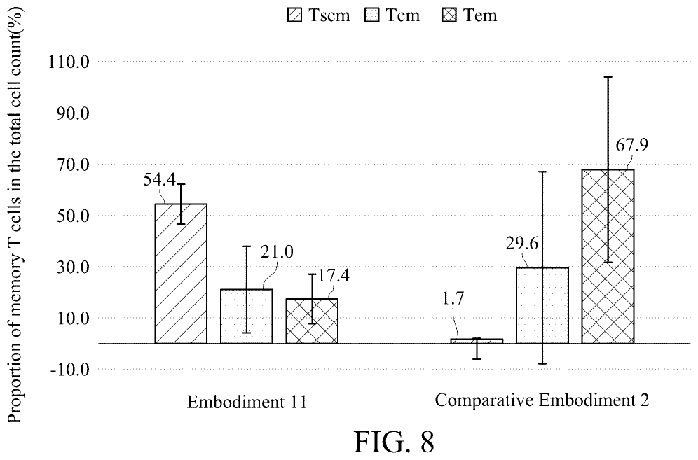

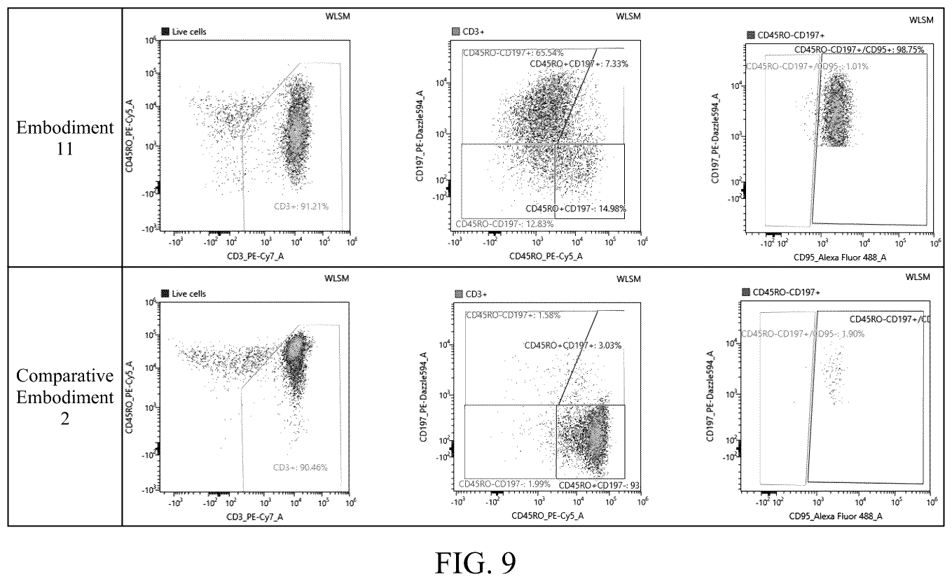

FIG. 8 shows the statistical analysis of the proportions of various memory T cells in the activated T cell count, averaged from multiple unshown batches, after batch cultured with the composition of the embodiment of the present disclosure and the composition of the comparative embodiment. FIG. 9 shows the flow cytometry analysis chart obtained after cultured with the composition of the embodiment of the present disclosure and the composition of the comparative embodiment. Table 10 discloses the components of the compositions used in this experiment for the embodiment and comparative embodiment, where comparative embodiment 2 is a composition disclosed in prior arts. FIG. 8, FIG. 9, and Table 11 show the proportions of stem cell memory T cells (Tscm), central memory T cells (Tcm), and effector memory T cells (Tem) in the activated T cell count of the second cell population. As shown in FIG. 8, FIG. 9, and Table 11, compared to the composition of comparative embodiment 2, culturing cells with the composition of embodiment 11 of the present disclosure results in a higher proportion of stem cell memory T cells (Tscm), and the combined proportion of stem cell memory T cells (Tscm) and central memory T cells (Tcm) is also higher.

TABLE 10
Components of each composition

| | |
|---|---|
| Embodiment 11 | 50 U/mL interleukin-7 |
| | 50 U/mL interleukin-15 |
| | 10 mM N-acetylcysteine |
| | 5% (v/v) human platelet lysate |
| Comparative | First phase (D0-D5): RPMI-1640 + 1 mM NA + |
| Embodiment 2 | pyruvate, 2 mM L-glutamine, 1 mM OAA, 0.2 U/ml |
| | insulin, 60 μg/ml Kanamycin, 10% human serum and |
| | 700 IU/ml IL-2 |
| | Second phase (D6-D14): AIM-V + 1 mM OAA, 1% |
| | human serum and 175 IU/ml IL-2 |

TABLE 11
Proportion (%) of each type of memory T cell
population in the activated T cell count

| | Tscm | Tcm | Tem |
|---|---|---|---|
| Embodiment 11 | 54.4 | 21.0 | 17.4 |
| Comparative Embodiment 2 | 1.7 | 29.6 | 67.9 |

### Experiment 7: Comparison of the Proportions of Activated T Cells, Natural Killer T Cells, and Cytotoxic T Cells with Comparative Embodiment

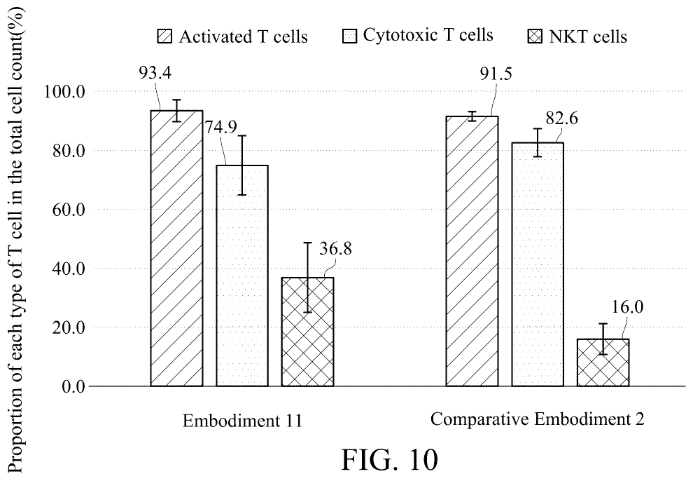

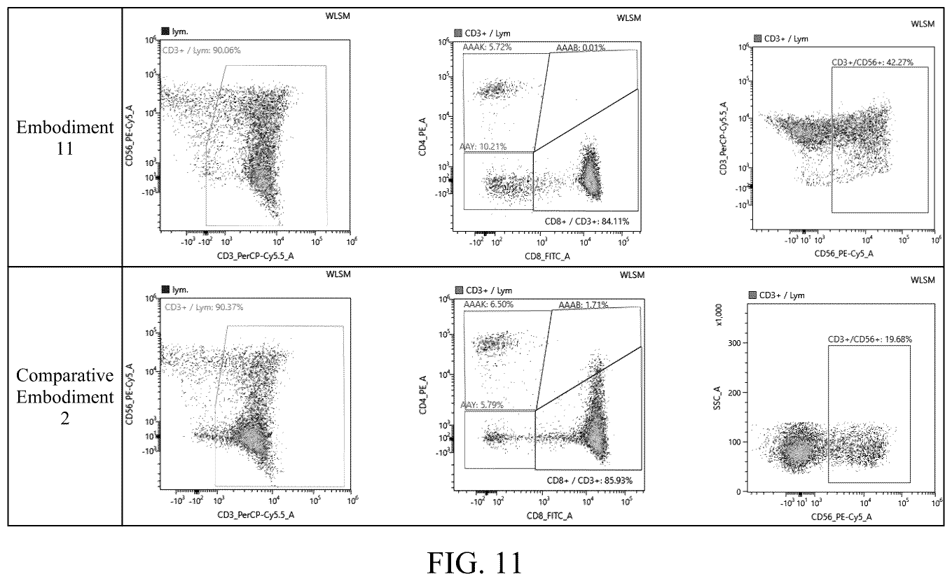

The embodiment and comparative embodiment (i.e., embodiment 11 and comparative embodiment 2) used in this experiment are the same as those in Experiment 6. FIG. 10 shows the statistical analysis of the proportions of various T cells in the total cell count, averaged from multiple unshown batches, after batch manufactured with the composition of the embodiment of the present disclosure and the composition of the comparative embodiment. FIG. 11 shows the flow cytometry analysis chart obtained after manufactured with the composition of the embodiment of the present disclosure and the composition of the comparative embodiment. FIG. 10 and Table 12 show the proportions of CD3+ activated T cells, CD3+CD56+ natural killer T cells (NKT cells), and CD3+CD8+ cytotoxic T cells in the total cell count of the second cell population. As shown in FIG. 10, FIG. 11, and Table 12, manufacturing cells with the composition of embodiment 11 of the present disclosure results in a high proportion of activated T cells, cytotoxic T cells, and natural killer T cells. Additionally, compared to the composition of comparative embodiment 2, culturing cells with the composition of embodiment 11 of the present disclosure results in a higher proportion of natural killer T cells.

TABLE 12
Proportion (%) of each type of T cell in the total cell count

| | Activated T | Cytotoxic T | NKT |
|---|---|---|---|
| | Cells | Cells | Cells |
| Embodiment 11 | 93.4 | 74.9 | 36.8 |
| Comparative Embodiment 2 | 91.5 | 82.6 | 16.0 |

### Experiment 8: Cytotoxic Efficiency Against Cancer Cells

The following experiment used impedance-based cytotoxicity assay technology to analyze the cytotoxic effect. In the impedance-based cytotoxicity assay, the target cells are cancer cell lines, and the effector cells are the cells manufacturing with the compositions. First, the impedance values are calibrated, and the target cells are cultured in a 96-well impedance plate, with their impedance values monitored. After 16 to 18 hours, effector cells are added at a specific ratio (Effector cell:Target cells, E:T) to induce cytotoxicity, and the impedance changes are observed to calculate the cytotoxic efficiency.

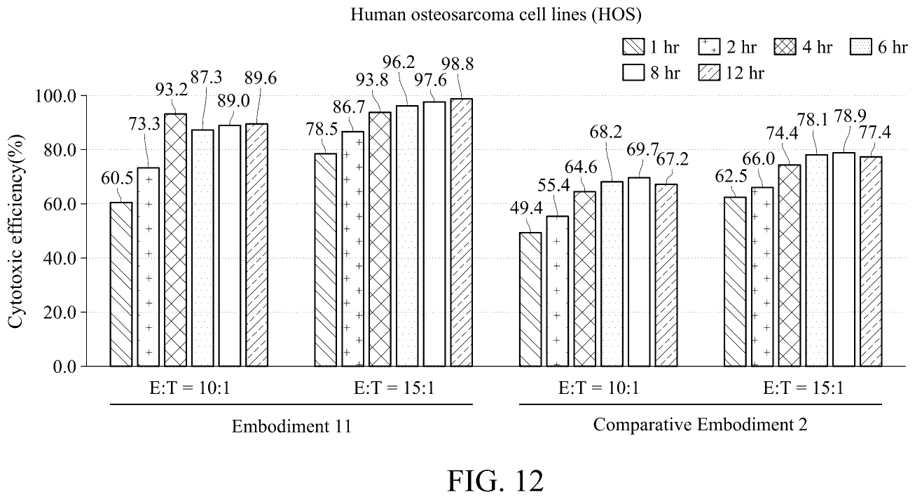

FIG. 12 shows the cytotoxic efficiency of the cells manufactured with the composition of the embodiment of the present disclosure and the composition of the comparative embodiment against osteosarcoma cancer cell lines at different time and different E:T ratio. The target cells are human osteosarcoma cell lines, HOS, and the effector cells are the cells manufactured with the composition of embodiment 11 of the present disclosure or the composition of comparative embodiment 2. As shown in FIG. 12 and Table 13, at the same E:T ratio or at the same cytotoxic duration, the cell population manufactured with the composition of embodiment 11 of the present disclosure demonstrates better cytotoxic efficiency against bone cancer cell lines compared to the composition of comparative embodiment 2.

TABLE 13
Cytotoxic efficiency(%)

| | | Embodiment 11 | | | Comparative Embodiment 2 | | |
|---|---|---|---|---|---|---|---|
| | | E:T = | E:T = | E:T = | E:T = | E:T = | E:T = |
| Duration | | 10:1 | 15:1 | 30:1 | 10:1 | 15:1 | 30:1 |
| 1 | hr | 60.49 | 78.50 | 91.59 | 49.41 | 62.46 | 75.83 |
| 2 | hr | 73.28 | 86.72 | 97.91 | 55.41 | 65.96 | 79.21 |
| 4 | hr | 93.19 | 93.75 | 100 | 64.56 | 74.36 | 84.26 |
| 6 | hr | 87.32 | 96.24 | 100 | 68.22 | 78.11 | 86.96 |
| 8 | hr | 88.95 | 97.55 | 100 | 69.7 | 78.94 | 87.96 |
| 12 | hr | 89.59 | 98.84 | 100 | 67.22 | 77.39 | 87.96 |
| 24 | hr | 88.48 | 100 | 100 | 57.25 | 68.08 | 84.45 |
| 36 | hr | 92.17 | 100 | 100 | 50.47 | 62.34 | 84.96 |
| 48 | hr | 96.98 | 100 | 100 | 45.4 | 58.05 | 86.29 |
| 60 | hr | 99.94 | 100 | 100 | 35.32 | 48.72 | 85.84 |
| 72 | hr | 100 | 100 | 100 | 27.92 | 41.05 | 86.78 |

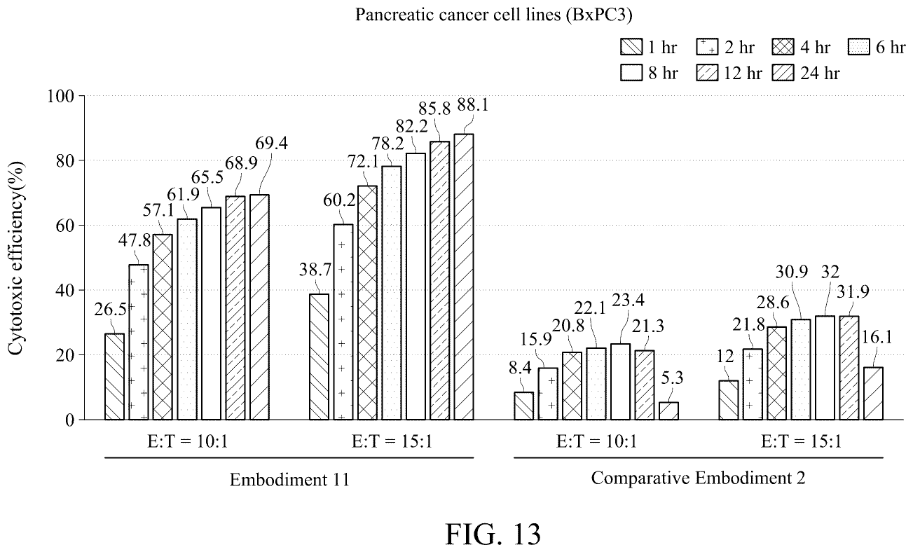

FIG. 13 shows the cytotoxic efficiency of the cells cultured with the composition of the present embodiment of the present disclosure and the composition of the comparative embodiment against pancreatic cancer cell line at different time and different E:T ratio. The target cells are human pancreatic cancer cell line, BxPC3, and the effector cells are the cells cultured with the composition of embodiment 11 of the present disclosure or the composition of the comparative embodiment 2. As shown in FIG. 13 and Table 14, at the same E:T ratio or at the same cytotoxic duration, the cell population manufactured with the composition of embodiment 11 of the present disclosure demonstrates better cytotoxic efficiency against pancreatic cancer cell line compared to the composition of comparative embodiment 2.

TABLE 14
Cytotoxic efficiency(%)

| | | Embodiment 11 | | | Comparative embodiment 2 | | |
|---|---|---|---|---|---|---|---|
| | | E:T = | E:T = | E:T = | E:T = | E:T = | E:T = |
| Duration | | 10:1 | 15:1 | 30:1 | 10:1 | 15:1 | 30:1 |
| 1 | hr | 26.46 | 38.73 | 54.94 | 8.38 | 11.98 | 13.55 |
| 2 | hr | 47.8 | 60.21 | 77.39 | 15.9 | 21.77 | 26 |
| 4 | hr | 57.12 | 72.06 | 90.62 | 20.8 | 28.63 | 36 |
| 6 | hr | 61.85 | 78.19 | 96.33 | 22.08 | 30.91 | 45.89 |
| 8 | hr | 65.53 | 82.21 | 99.0 | 23.42 | 31.96 | 54.37 |
| 12 | hr | 68.91 | 85.82 | 100 | 21.34 | 31.9 | 59.68 |
| 24 | hr | 69.38 | 88.07 | 100 | 5.28 | 16.1 | 58.44 |
| 36 | hr | 56.18 | 87.97 | 100 | 0 | 0 | 46.32 |
| 48 | hr | 50.03 | 87.24 | 100 | 0 | 0 | 35.33 |
| 60 | hr | 38.24 | 85.39 | 100 | 0 | 0 | 21.85 |
| 72 | hr | 19.91 | 80.72 | 100 | 0 | 0 | 12.18 |

From the aforementioned experiments, it may be concluded that the cell population obtained by manufacturing with the composition of the embodiments of the present disclosure contains a high proportion of stem cell memory T cells, is also rich in activated T cells, natural killer T cells and cytotoxic T cells, and exhibits excellent cytotoxic effects against cancer cells.

As described above, according to the composition and method for ex vivo expansion of stem cell memory T cells in the embodiments of the present disclosure, the obtained cell population has a high proportion of stem cell memory T cells. Moreover, compared to the obtained cell population from compositions disclosed in prior arts, the cell population obtained by using the composition of the embodiments of the present disclosure has a higher proportion of stem cell memory T cells, as well as a higher combined proportion of stem cell memory T cells and central memory T cells. In addition, according to the composition and method for ex vivo expansion of stem cell memory T cells in the embodiments of the present disclosure, the obtained cell population also has a high proportion of natural killer T cells. Moreover, compared to the obtained cell population from compositions disclosed in prior arts, the cell population obtained by using the composition of the embodiments of the present disclosure exhibits superior cytotoxic effects against bone and pancreatic cancer cells. Therefore, the composition and method for ex vivo expansion of stem cell memory T cells in the embodiments of the present disclosure may obtain a cell population with a high proportion of stem cell memory T cells, improve the production efficiency of stem cell memory T cells, and hold great potential for use in cancer treatment or be used as a raw material for chimeric antigen receptor T cell (CAR-T) therapy.

What is claimed is:

1. A composition for ex vivo expansion of stem cell memory T cells, comprising:
25 U/mL to 50 U/mL of interleukin-7 or 0.192 ng/mL to 25 ng/mL of interleukin-7;
10 U/mL to 50 U/mL of interleukin-15 or 0.5 ng/mL to 25 ng/mL of interleukin-15;
1 mM to 10 mM of N-acetylcysteine; and
0% to 10% (v/v) of human platelet lysate.

2. A method for ex vivo expansion of stem cell memory T cells, comprising:
manufacturing a first cell population by using the composition for ex vivo expansion of stem cell memory T cells of claim 1 to obtain a second cell population,
wherein the first cell population comprises CD3+ activated T cells, CD3+CD45RO−CD197+CD95+ stem cell memory T cells, CD3+CD45RO+CD197+ central memory T cells, CD3+CD45RO+CD197− effector memory T cells, CD3+CD56+ natural killer T cells, and CD3+CD8+ cytotoxic T cells, the second cell population comprises CD3+ activated T cells, CD3+CD45RO−CD197+CD95+ stem cell memory T cells, CD3+CD45RO+CD197+ central memory T cells, CD3+CD45RO+CD197− effector memory T cells, CD3+CD56+ natural killer T cells, and CD3+CD8+ cytotoxic T cells, and the proportion of CD3+CD45RO−CD197+CD95+ stem cell memory T cells in a activated T cell count of the second cell population is 35.6% or higher.

3. The method for ex vivo expansion of stem cell memory T cells of claim 2, wherein the proportion of CD3+CD56+ natural killer T cells in a total cell count of the second cell population is 17.1% or higher.

4. The method for ex vivo expansion of stem cell memory T cells of claim 2, wherein the proportion of CD3+CD8+ cytotoxic T cells in a total cell count of the second cell population is 64.2% or higher.

5. The method for ex vivo expansion of stem cell memory T cells of claim 2, wherein the proportion of CD3+CD45RO−CD197+CD95+ stem cell memory T cells in the activated T cell count of the second cell population ranges from 35.6% to 64.7%.

6. The method for ex vivo expansion of stem cell memory T cells of claim 2, wherein the proportion of CD3+CD56+ natural killer T cells in a total cell count of the second cell population ranges from 17.1% to 55.8%.

7. The method for ex vivo expansion of stem cell memory T cells of claim 2, wherein the proportion of CD3+CD8+ cytotoxic T cells in a total cell count of the second cell population ranges from 64.2% to 93.3%.

8. The method for ex vivo expansion of stem cell memory T cells of claim 2, wherein a culture temperature is 37° C., and a manufacturing time ranges from 3 to 6 days.
